(19) United States
(12) Patent Application Publication
(10) Pub. No.: US 2021/0272058 A1
(43) Pub. Date: Sep. 2, 2021

(54) METHOD AND SYSTEM TO DETECT DRUG DELIVERY

(71) Applicant: Lampros Kourtis, Cambridge, MA (US)

(72) Inventor: Lampros Kourtis, Cambridge, MA (US)

(73) Assignee: Lampros Kourtis, Cambridge, MA (US)

(21) Appl. No.: 17/255,639

(22) PCT Filed: Jun. 25, 2019
(86) PCT No.: PCT/IB2019/055320

§ 371 Date: Dec. 23, 2020

Related U.S. Application Data
(60) Provisional application No. 62/689,953, filed on Jun. 26, 2018.
(60) Provisional application No. 62/690,231, filed on Jun. 26, 2018.

(51) Int. Cl. G06Q 10/08; A61J 7/00; A61J 1/03; G16H 20/10; G06N 5/02

(52) U.S. Cl. CPC G06Q 10/0835; A61J 7/0084; A61J 1/035; A61J 2205/60; G06N 5/02; A61J 2200/30; A61J 2200/70; G16H 20/10

(57) ABSTRACT

Medication adherence monitoring is a long standing problem in the healthcare system. The present disclosure describes a method and a system to detect and confirm drug delivery as intended by a combination of a) smart medication packaging, that annotates the cloud when its contents are released and b) a pattern recognition algorithm to processes wearable and mobile device signals that signify the action of a subject receiving said medication. Embodiments of the method and system apply to daily dose packaging using pouches or blister pack packaging or liquid drug packaging. The disclosure further describes elements of transmission of the adherence information to the members of the healthcare ecosystem.

### FIELD OF THE INVENTION

The present invention relates generally to the field of medication adherence monitoring and medication adherence improvement. More particularly, the invention refers to the field of packaging state detection in combination with the field of activity tracking and characterization in the context of medication delivery. Packaging state detection hereby refers to confirmation of a package of an oral, inhalable or injectable medication being opened or not, hence a drug being dispensed. Activity tracking and characterization hereby refers to the use of artificial intelligence to identify patterns that are related to the action of receiving medication. The invention also refers to means of improving medication adherence by assisting patients with determining and reminding of their drug dosing.

### BACKGROUND OF THE DISCLOSURE

Despite efforts undertaken by Pharma, Payers, and Healthcare Practitioners, adherence to medication remains an unresolved problem with multiple public health, economic, and health outcomes effects. One step to improve medication adherence is to monitor it. To date, a plurality of methods has been disclosed to help detect drug delivery. U.S. Pat. No. 7,081,807B2 discloses a smart pill bottle that has a timer incorporated on the cap, and hence can alert the user for the next dose. US20070016443A1 describes a method and system that actively monitors patient's compliance using medication containers, or pill caps that wirelessly communicate with a server and inform a compliance monitoring database each time a patient has used the device. U.S. Pat. No. 9,235,683B2 discloses a system and a method the relies in the combination of ingestible device that produces an event after ingestion that is traced by an apparatus outside of the body that further communicates the ingestible event with a drug compliance monitoring database.

### SUMMARY

Medication can be packaged in one of the following forms, box, blister pack, individual blisters, sachets, sachet roll, pouch roll, pouches, individual bags. The present invention provides a method and a system to detect a) when one the abovementioned packaging forms has been opened so that the medication is released to the subject and b) when the subject has actually received the medication that was previously released from its packaging. The first is achieved by incorporating sensing or detectable features on the packaging. The later is achieved using pattern recognition techniques that monitor a plurality of sensors signals (movement, sound, proximity, location) including signals from wearable sensors that indicate a pattern that has been previously correlated with the action of receiving a medication. Drug delivery confirmation can be inferred from the detection of the packaging being opened in combination with said patterns being detected as well as other user, contextual and environmental factors.

### BRIEF DESCRIPTION OF THE DRAWINGS

FIG. 1 depicts an oral medication (1) packaged in a pouch roll (2) that is comprised of multiple compartments, or pouches, or sachets (3) having a printed circuit (4) that is comprised of a set of resistors (6) that are connected by connecting lines (7). Pouches or sachets (3) are torn, opened or removed from the roll (2) at predetermined positions marked by a crease (8) that divides each pouch or sachet (3). A control unit (5) senses an electrical property along the connecting lines (7), in this embodiment it is resistance.

FIG. 2a shows one embodiment of the invention in which the roll (2) is comprised of pouches or sachets (3) that have an embedded or printed circuit (4) of resistors (6) corresponding to each pouch or sachet (3) and are connected in parallel (7) and can have any value Rk. The total resistance Rtotal is measured by the control unit (5). Each pouch or sachet (3) can be removed by tearing across the perforation line (8). FIG. 2b represents the resistance of each resistor Rk of each pouch or sachet (3) where Rk is such that the drop in the total circuit resistance Rtotal is proportional to the ratio of the resistors removed divided by the total number of resistors N.

FIG. 3 shows another embodiment of the invention in which the roll (2) is comprised of pouches or sachets (3) that have an embedded or printed circuit (4) of capacitors (9) connected in parallel with two conductive connecting lines (7). Upon tearing or cutting or removal of part of the sheet/roll at the perforation lines (8), one or more capacitors belonging to the torn pouch(es) or sachet(s) (10) are removed from the circuit (4) and the total capacitance changes. The overall capacitance of the circuit is measured by a control unit (5).

FIG. 4 shows another embodiment of the invention in which the roll (2) is comprised of pouches or sachets (3) that have an embedded or printed circuit (4) of inductors (11) connected in parallel with two conductive lines (7). Upon tearing or cutting or removal of part of the sheet/roll at the perforation lines (8), one or more inductors belonging to the torn pouch(es) or sachet(s) (10) are removed from the circuit (4) and the total inductance changes. The overall inductance of the circuit is measured by a control unit (5).

FIG. 5 shows another embodiment of the invention in which the roll (2) is comprised of pouches or sachets (3) that have an embedded or printed circuit (4) that is a comprised of a sub-circuit of resistors and/or capacitors and/or inductors (RLC) (12), connected in parallel with two conductive connecting lines (7). Upon tearing or cutting or removal of part of the sheet/roll at the perforation lines (8), one or more RLC sub-circuits (12) belonging to the torn pouch(es) or sachet(s) (10) are removed from the circuit (4) and the total impedance changes. The overall impedance of the circuit is measured by a control unit (5).

FIG. 6 describes a control unit (5) that is connected with the printed or embedded circuit (4) of the roll (2). The control unit (5) is comprised of a data acquisition system (DAQ) (13), a processor and memory system (14) and a communication system (15). The control unit (5) is able to communicate with a handheld device or a connected device (16) such as a smartphone, smartwatch or any other connected devices that is able to receive signals generated by the communication system (15). The communication system (15) transmits the events acquired from the pouch roll (2), such as removal of a pouch (10), to the internet (17). A server (18) is processing and storing the events and presents analyzed data to a terminal front-end (19) that is connected to the internet.

FIG. 7 shows a portion of a roll (2) that has at least 3 pouches (3) each one containing a medication dose (1). The roll (2) has the two connecting lines (7) running across its length and each pouch holds an impendence element (12). Each pouch (3) is separated from the next one by a perforation line (8) and holds a crease (22) for opening it. A control unit (5) measures the impendence across the connecting lines (7) and transmits the state of the roll to the cloud. Once a pouch is removed from the roll (2) an RFID circuit (21) transmits its state: unopened (10) when the impendence element is part of the circuit or opened (20) when the impendence element is interrupted from the circuit due to the tear (30) across the crease (22). The RFID circuit (21) is comprised of an RFID chip (47) and an RFID antenna (24).

FIG. 8 shows a variation of the embodiment shown in FIG. 7 with a portion of a roll that has at least 3 pouches each one containing a medication dose (1). The roll has the two connecting lines running across its length and each pouch holds an impendence element (12). Once a pouch is removed from the roll an RFID circuit (21) transmits its state: unopened (10) when the impendence element is part of the circuit or opened (20) when the impendence element is interrupted from the circuit (21) due to the tear (30) across the crease (22).

FIG. 9 shows a portion of a roll (2) that has at least 3 pouches (3) each one containing a medication dose (1). The roll (2) has the two connecting lines (7) running across its length and each pouch holds an impendence element (12). Each pouch (3) is separated from the next one by a perforation (8) and holds a crease (22). Each pouch holds two resonating antennas (25 and 26) that are connected using a bridge line (27). Upon tearing (30) of the pouch (10 or 20), the bridge line (27) is interrupted, hence the resonant frequency of the circuit changes signaling a change of state from unopened (10) to opened (20).

FIG. 10 shows a portion of a roll (2) that has at least 4 pouches (3) each one containing a medication dose (1). Each pouch (3) is separated from the next one by a perforation (8) and holds a crease (22). Each pouch holds a plurality of resonators (28) whose pattern is unique for each pouch. Resonators are printed on placeholder patterns (28 and 29). Dark lines signify the resonators present (28) in a particular pouch (10) and dotted or gray lines signify the resonators missing (29) or the placeholders, from that particular pouch (10). Upon tearing (30) of the pouch, some resonators are interrupted (31) while some are not interrupted (32).

FIG. 11 shows a spectrum (33) of a pouch, with the resonant frequency peaks (34) of the resonators (28) shown in solid line as well as the frequency peaks (35) of placeholder resonators (29) not present in this example.

FIG. 12A shows the back side of a blister-pack (36) with its backing film (37) and its blisters (40). The connecting lines (7) connect resistive elements (6), hereby with dashed lines, that get interrupted when a perforation occurs on the film (37) due to the medication (1) being pushed out through the perforation (39) in the backing film (37). A control unit (5) that holds a data acquisition system (13), a processing and memory system (14) and a communication system (15) measures changes in resistance across the connecting lines (7) and hence detects a change of state for a blister (40). This information is transmitted to the cloud. FIG. 12B shows a schematic of a circuit (4) that is printed on the backing film (37) of a blister-pack (36) with the connecting lines (7) and the resistive elements (6) as well as a foil perforation (39) and an oral medication (1) being released from its blister (40).

FIG. 13 shows a portion of a roll (2) with pouches that each contain medication (1) and have each a resistor element (6) as well as a plurality of resonators (28). At the end of the roll, a control unit (5) comprises a Data/Signal Acquisition System (13), a Processing and Memory system (14), and a communication system (15) that is capable of communicating with a mobile or wearable device (41) using Bluetooth or Wi-Fi protocols. The control unit (5) measures the resistance across the connecting lines (7) and transmits the state of the roll (2) to the cloud. Once a pouch is removed from the roll, a wearable device (41) can probe the state of the pouch (10). The wearable device (41) can probe the resonators (28) that are printed on the surface of the pouch (10 or 20) material. When a tear (30) happens, some of the resonators are interrupted (31), hence their frequency peak disappears, whereas, some resonators do not get interrupted, hence their peaks remain and serve as a communication reference.

FIG. 14 shows a subject (42) who is wearing a smart watch (43) taking an oral medication (1) and the trajectory (44) his/her hands (45) follow in the process. The smart watch (43) holds a 9 Degree of Freedom (DOF) Inertial Moment Unit (IMU) comprised of accelerometer, gyroscope and magnetometer. The IMU can accurately represent the trajectory and rotational position of the wearable at a given time by using sensor fusion and transformation techniques with a priori knowledge, such as prior knowledge filtering: signal integration and Kalman filtering. The trajectories (44) of the hand (45) as well as the oral medication (1) are estimated by overlaying anthropometric data.

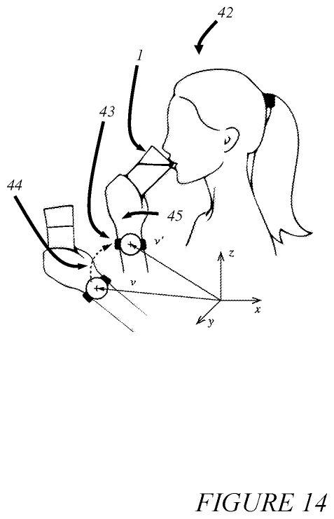

FIG. 15 shows a trajectory (44), in Cartesian coordinates, that is created when a user takes an oral medication. In one scenario, the hand carrying the pill is the one that carries the wearable—smart ring or smart watch as well. In another scenario, the hand carrying the cup of water or drink is the one that carries the wearable smart ring or smart watch. In both cases, the hand trajectory has in principle a unique pattern in terms of spatial characteristics as well as in terms of velocity and acceleration that can be used by an pattern recognition algorithm to detect drug delivery.

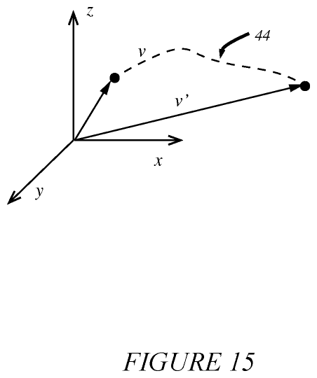

FIG. 16 shows a subject (42) who is wearing a smart watch (43) taking an oral medication (1) in a home setup and the sources of sound waves (48) that are produced in the process. The medication package opening sound, tap water sound, the cup filling sound, the gulping sound as the oral medication is being swallowed are captured by the smart watch's microphone (46). This sound sequence presents a unique pattern that can used by an pattern recognition algorithm to detect drug delivery.

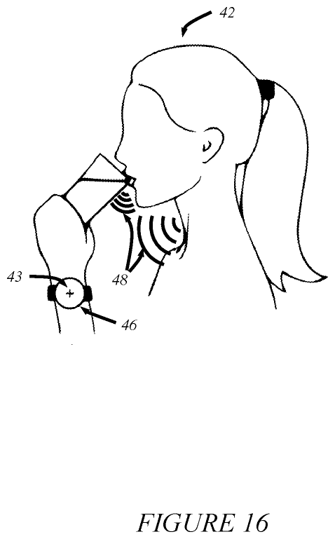

FIG. 17 shoes a plot of the accelerations (49) that are recorded from a wearable device as a subject wearing it is bringing oral medication (1) to his mouth with the purpose of consuming it. All three components of the acceleration can be used to reconstruct the actual path that the wearable is following.

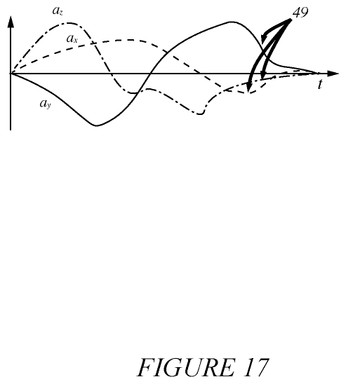

FIG. 18 shows a schematic of the potential sources of signals that can be combined into a pattern recognition algorithm (50) that can provide a probability of medication being delivered as intended. These signals are sound, movement (acceleration, rotation, magnetic orientation), location, time/date, delivery device analog or digital signals (for example package open or not open), prescription info and demographics and epidemiology info.

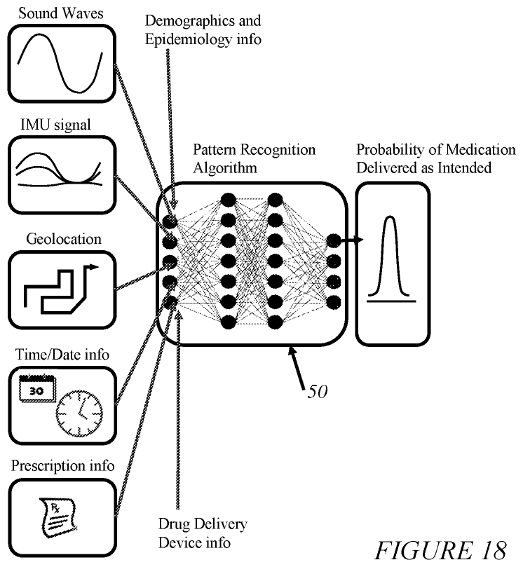

FIG. 19 shows the continuum of the invention with the packaging in a roll (2) that holds multiple pouches and a printed circuit (4) with each pouch having a resistive element. The control unit that comprises a data/signal acquisition system (13), a processing and memory system (14) and a communication system (15) detects a tearing or pouch removal event and signals the cloud through a connected device (16). At the time when the subject (42) receives her medication (1), the movement trajectory (44) of her hand (45) as well as any sounds (48), captured by the wearable device (43) sensors and. An algorithm (50) that is installed on a program on the wearable (43) or mobile device (16) estimates the probability that a pattern correlates with the action of taking an oral medication based on prior training. The wearable device (43) communicates this probability to a connected device (16). The connected device (16) transmits both the packaging state and the probability that the subject has performed the recognized steps in taking her medication to the cloud. A server (18) processes the information and sends to a front end (19).

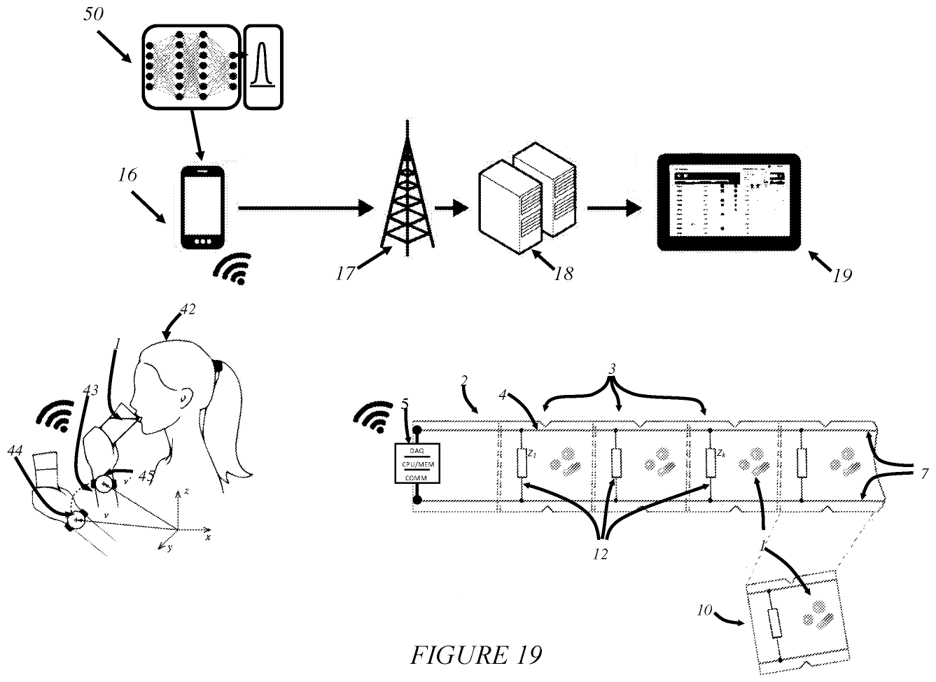

### DETAILED DESCRIPTION

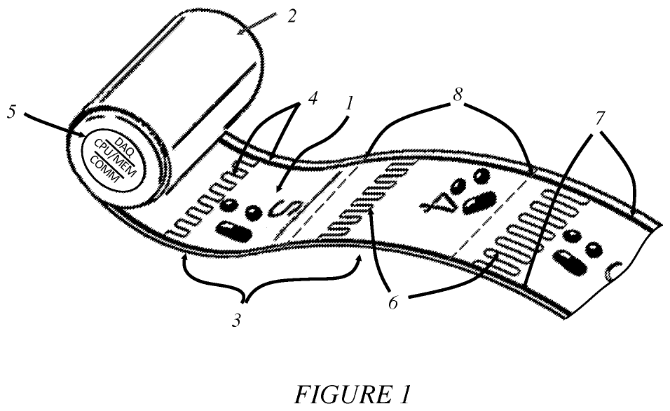

In one embodiment, oral medication (1) is packaged in a pouch roll (2) that is capable to deliver daily doses as shown in FIG. 1. The roll (2) may hold compartments, or pouches, or sachets (3) that contain pharmaceutical substances that are prescribed for a particular subject. The roll (2) holds a circuit (4) that is embedded or printed on its surface. The circuit (4) can be printed on the sheet/roll using conductive ink, or can be printed using lithographic methods, or can be cut from an original sheet of conductive and/or or resistive material and can be laminated with the sheet/roll. Every time that a compartment or pouch or sachet (3) is torn open or removed from the roll (2), the electric properties of the circuit (4) change and can be detected by a control unit (5).

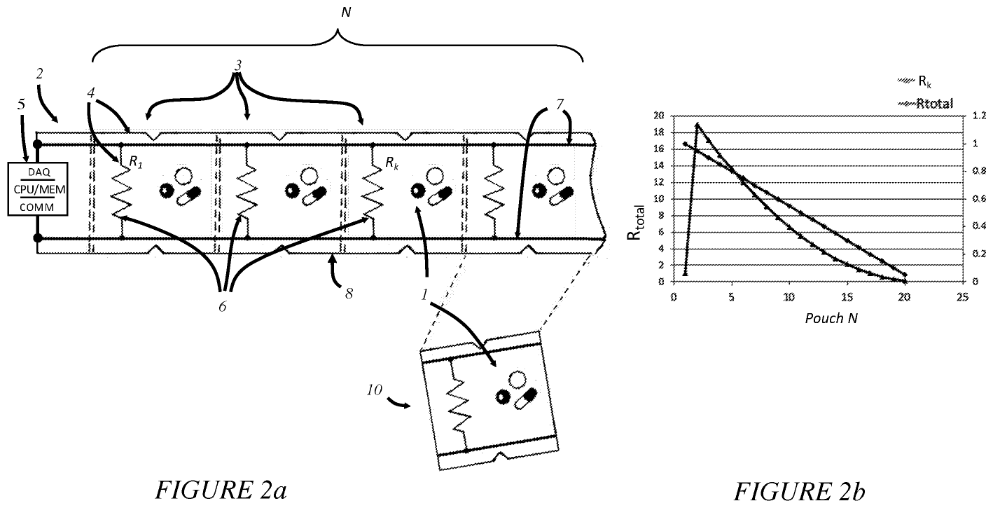

In one embodiment, the roll or pouch or sachet material has an embedded circuit of resistors (6) in parallel as shown in FIGS. 1 and 2.). Each pouch or sachet (3) is separated by a crease (8). Upon tearing or cutting or removal or cracking of part of the sheet/roll at the creased sites (8), one or more resistors (6) are removed from the circuit (4), therefore the total resistance across the connecting lines (7) of the circuit (4) changes. The overall resistance of the circuit (4) is measured by a control unit (5). Two connecting conductive lines (7) run longitudinally along the roll (2) to connect all the resistors (6) in parallel.

In one embodiment, the resistance of each resistor Rₖ (6) of each pouch or sachet (3) is selected such that the drop in the total circuit resistance can be proportional to the ratio of the resistors removed divided by the total number of resistors. Each of the resistors (6) that are connected in parallel using the connecting lines (7) can have any value, but in one embodiment, the resistance Rₖ of the k^{th} resistor in a total of N resistors has a value so that the drop in the total resistance along the connecting lines (7) of the circuit (4) is proportional to the number of the resistors removed and is given by the following equation as shown in FIG. 2b for N=20:

$${R}_{k} = \frac{{R}_{1}}{\frac{N}{N - k + 1} - \frac{N}{N - k + 2}}$$

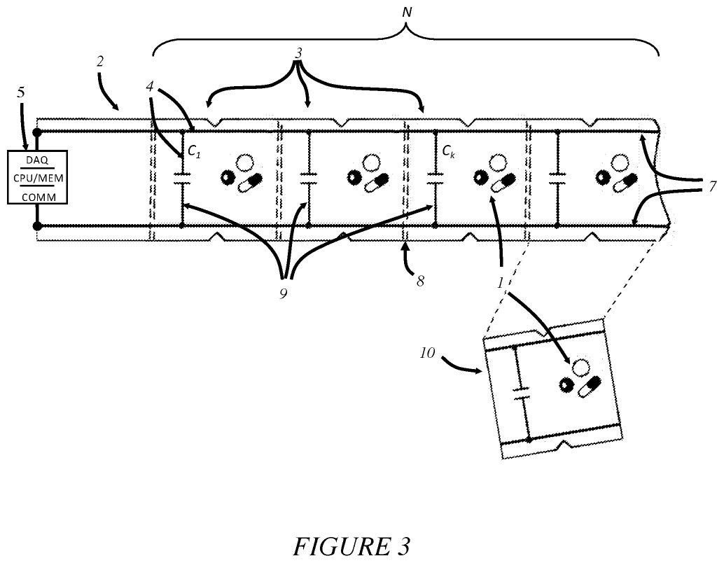

In one embodiment shown in FIG. 3, the sheet/roll (2) has an embedded or printed circuit (4) of capacitors (9) in parallel. The circuit can be printed on the sheet/roll using conductive, resistive and/or dielectric ink, or can be printed using lithographic methods, or can be cut from an original sheet of conductive and/or dielectric material and can be laminated with the sheet/roll. Upon tearing or cutting or removal of part of the sheet/roll at the creased sites (8), one or more capacitors (9) are removed from the circuit (4), therefore the total capacitance across the connecting lines (7) of the circuit (4) changes. The overall capacitance of the circuit (4) is measured by a control unit (5). Two conductive connecting lines (7) run longitudinally along the roll to connect all the capacitors in parallel. By selecting the resistance of each capacitor Cₖ, the drop in the total circuit capacitance can be proportional to the ratio of the capacitors removed along with the removed pouch (10) divided by the total number of the remaining capacitors (9). In one embodiment, the capacitance of each capacitor is given by the following equation:

Cₖ=Cₖ₋₁

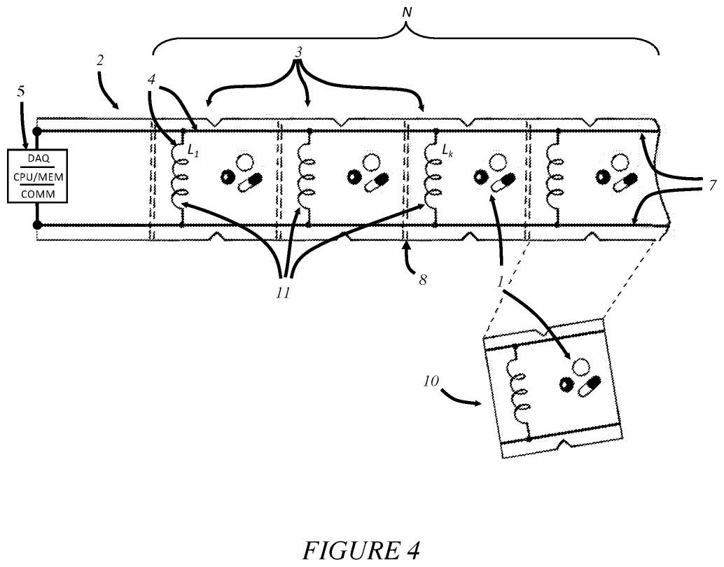

In one embodiment shown in FIG. 4, the sheet/roll (2) has an embedded or printed circuit (4) of inductors or coils (11) in parallel. The circuit can be printed on the sheet/roll using conductive ink, or can be printed using lithographic methods, or can be cut from an original sheet of conductive or resistive material and can be laminated with the sheet/roll. Upon tearing or cutting or removal of part of the sheet/roll at the creased sites (8), one or more inductors (11) are removed from the circuit (4), therefore the total induction across the connecting lines (7) of the circuit (4) changes. The overall inductance of the circuit is measured by a control unit (5). Two conductive lines (7) run longitudinally along the roll (2) to connect all the inductors (11) in parallel. By selecting the inductance of each inductor Lₖ, the drop in the total circuit inductance can be related to the ratio of the inductors removed along with the removed pouch (10) in relation to the total number of inductors. In one embodiment, the inductance of each inductor (11) is given by the following equation:

$${L}_{k} = \frac{{L}_{1}}{\frac{N}{N - k + 1} - \frac{N}{N - k + 2}}$$

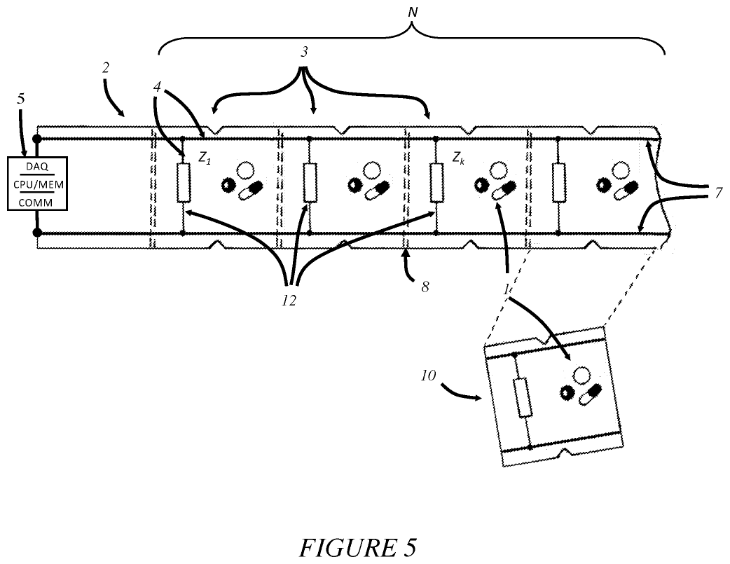

In one embodiment shown in FIG. 5, the sheet/roll (2) has an embedded circuit (4) of sets of resistors and/or capacitors and/or inductors or combinations thereof comprising LRC or RL or RC or LC sub-circuits (12) that are connected in parallel. The circuit (4) can be printed on the sheet/roll using a combination of conductive, resistive and/or dielectric ink, or can be printed using lithographic methods, or can be cut from an original sheet of conductive or resistive or dielectric material and can be laminated with the sheet/roll. Upon tearing or cutting or removal of part of the sheet/roll (2) at the creased sites (8), one or more sub-circuits (12) are removed from the circuit (4), therefore the total impedance across the connecting lines (7) of the circuit (4) changes. The overall impedance of the circuit is measured by a control unit (5) that holds an impedance meter or an LRC meter. Two parallel conductive connecting lines (7) run longitudinally along the roll (2) to connect all the sub-circuits (12) in parallel. By selecting the impedance and the resonant frequency of each sub-circuit Zₖ, the drop in the total circuit impedance can be related to the number of the sub-circuits removed along with the removed pouch(es) (10) as shown in FIG. 5. In one embodiment, the impedance of each sub-circuit is given by the following equation:

$${Z}_{k} = \frac{{Z}_{1}}{\frac{N}{N - k + 1} - \frac{N}{N - k + 2}}$$

The circuit can be printed on the roll film using resistive and/or conductive ink, or can be printed using lithographic methods, or can be printed using inkjet printing, or can be printed using gravure printing, or can be printed using impression printing, or can be printed using screen printing, or can be printed using aerosol printing, or can be printed using flexography, or can be stamped, or can be printing using offset printing, or can be cut from an original sheet of conductive or resistive material and can be laminated with the roll. In one embodiment, the circuit may be incorporated in the roll film by means of conductive elements such as metal wires or metal or resistive strips that are assembled in the film. In one embodiment, the circuit may be created by eroding portions of a metal backed film using laser ablation or metal etching techniques or other stamping techniques, creating thus conducting channels that form said circuit.

The film that comprises the roll or pouch or sachet material may be comprised of PET, PVC, Polyurethane, Ethylene Vinyl Alcohol, polyethylene naphthalate, Polyimide, Mylar, Polyethylene (PE), Flashspun nonwoven HDPE fiber, TYVEK®, Polypropylene (PP), Polycarbonate or acrylic, or multi-layer combination thereof such as PET-PE or combinations thereof in laminate layers or polymer mixtures and metal coated variations of the above. In one embodiment, said film may be comprised of metal including aluminum, or polymer backed aluminum. In one embodiment, said film may be comprised of paper or plastic reinforced paper or metal reinforced paper.

The conductive ink may be comprised of silver, copper, nickel, silver chloride, carbon, copper, graphene, indium tin oxide, or polyaniline or Poly(3,4-ethylenedioxythiophene)-poly(styrenesulfonate) or/and combinations thereof.

In one embodiment, the circuit (4) that is embedded or printed or attached to the roll (2) is connected to a control unit (5) that holds a signal/data acquisition system (13) that can measure either the total a) resistance, or b) capacitance, or c) induction or d) impedance of the circuit. The control unit (5) comprises a data acquisition system (13) that allows for intermittent or continuous measurement of a circuit metric across the connecting lines (7) such as the a) resistance, or b) capacitance, or c) induction or d) impedance or combinations thereof. The data acquisition system (13) is connected to a processing and memory system (14) that has an algorithm that allows it to detect the detachment one or more compartments or pouches or sachets (3) from the roll (2), by comparing the rate of change of said metric over time. In one embodiment this rate of said metric is a comparison between dM/dt, where M is a measure, either of resistance R, capacitance C, induction L or impedance Z and c, a threshold value. When the condition below

dM/dt>c,

holds, then the processing system (14) detects a detachment of one or more components (10) from the roll (2). This condition can be used to detect serial detachment events in conjunction with a proportional drop in the total resistance or capacitance or inductance or impedance across the connecting lines (7) of the circuit (4) that is setup to comprise resistors (6) in parallel as indicated in equation (1). FIG. 2 shows a portion of a roll (2) with consecutive segments or interconnected sachets (3), along with a segment that has been detached (10), resulting in a change in the total resistance across the lines (7) of the circuit (4). The two ends of the parallel running leads are connected to the data acquisition system (13) that in turn is connected to the processing system (14). The data acquisition system (13) system also has a way to correct said metric values for temperature, proximity, electromagnetic interference, common mode or other environmental factors.

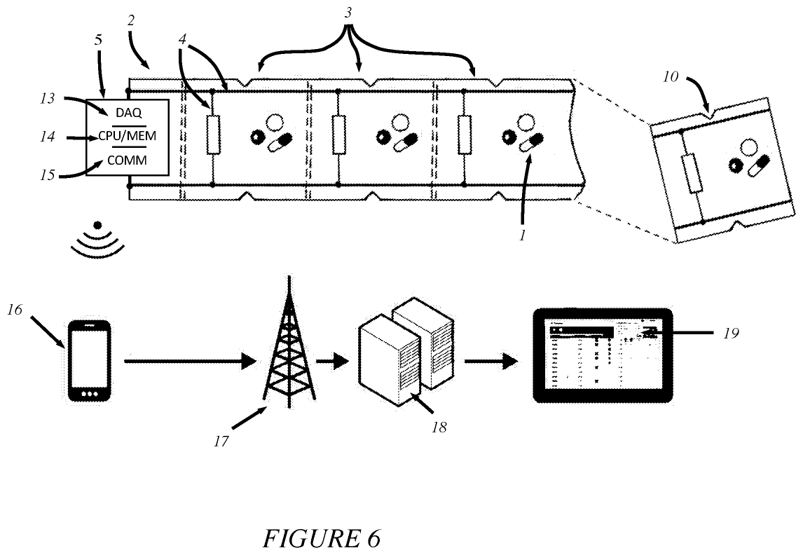

In one embodiment, the processing system is connected to a communication system (15) that is responsible for pairing and transmitting information to an external device or the cloud. The communication module can employ Bluetooth or Wi-Fi or RF or ultrasound or a combination thereof to transmit the information to a mobile device or another networking system such as a Wi-Fi router. The information is then transmitted and stored in the cloud so that it can further be processed. FIG. 6 shows the schematic of this continuum.

The abovementioned embodiments offer the capability to detect when an individual compartment (3) is removed or torn open from a roll (2) that holds multiple of them. Depending on the user preferences one or more compartments/sachets (3) may be removed from the roll (2) and stored for later use. This means that a sachet (3) is removed from the roll (2) but is not opened, hence the payload (the medication) is not delivered. The abovementioned embodiments can still signal that the sachet (3) has been removed from the roll, however, they cannot detect that the sachet (3) has actually been torn open to release the oral medication. Some of the next embodiments address this need, by incorporating passive or active sensing elements on each sachet (3) so that their state can be detected even after they have been removed from their original roll (2).

In one embodiment, the medication packaging is individual sachets or pouches comprised of a thin polymeric, metal coated polymeric, paper or foil film. Each separated sachet holds an RFID circuit that can be used to identify it and probe its state, meaning opened or unopened. The separated sachet (10) may or may not originally belong to a roll from which it can be detached. A series of packaging state detection methods that employ methods of RF communication are presented hereby.

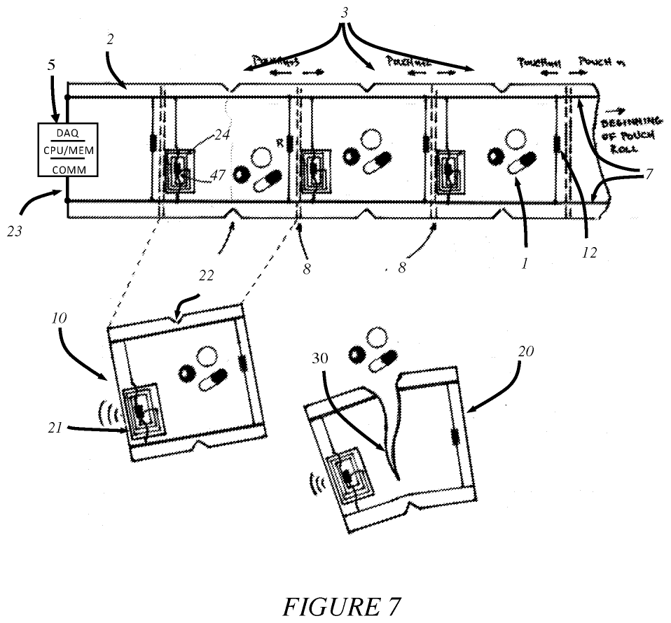

In one embodiment, shown in FIG. 7 the pouch roll (2) is comprised of multiple pouches (3) in series. Each pouch (3) contains the medication (1) that may be comprised of multiple oral drugs, pills, capsules, tablets or vials, syringes, cartridges. Each pouch (3) holds an impendence element (12) that can be a resistor, a capacitor or an inductor or a combination thereof (RLC circuit). These impendence elements are connected to conductive or resistive lines (7) that run across the entire roll (2) and connect the sub-circuit of each individual pouch. Removal of one pouch (10) from the roll (2) results in a change in the overall impendence between the two connecting lines (7) that can be measured using a control unit (5) connected at any point along the connecting lines (7). Each pouch (3) is separated from the next one by means of a crease or partial perforation (8). In one embodiment, the packaging may hold a RFID circuit (21) that contains at least one induction coil/antenna (24) or antenna and at least one identification chip (47). The chip (47) can a) collect the induction current generated on the RF circuit (21) by means of induction caused by an interrogation device (41) and b) modulate it in a way to produce a serialization code and status information code based on a sensing circuit printed on the package or sachet (3) and c) relay it back to the induction circuit (21) with the appropriate delay for re-emission so that it can be picked up by the interrogation device (41). This principle is similar to RFID technologies, with the addition of encoding further information with regards to the status of the package based on interruption of elements of the circuit, such as an open/unopened pouch, or liquid quantity remaining in a vial. The battery-less chipped RFID circuit (21) can detect a change in the state, unopened (10) and opened (20), of an individual pouch after said separated pouch (10 or 20) has been removed from the roll (2). The detection of the state of the pouch (opened/unopened) is done by means of a circuit interrupted on the pouch (20). When the subject opens the pouch (20) by tearing along the individual pouch crease (22), the circuit that runs along the perimeter of the pouch is interrupted. This change in the circuit of the pouch can be sensed by the RFID circuit (21) by means of switching circuit. The RFID circuit comprises an RFID chip (47) and an RF antenna (24) as shown in FIG. 7. This embodiment offers dual functionality,
A. when the pouch is attached to the roll, this design offers wired sensing of its state, that is done by means of sensing impedance (or resistance or capacitance or induction or combination thereof) between lines (7) at the end of the roll (23); said impedance changes when a pouch (3) has been opened to receive the payload and
B. when a pouch (10) has been removed from the roll (2) but has not been opened yet, this design offers wireless sensing of its state by means of circuit interruption when pouch is opened along the crease line (22) and transmittal of the state (opened/unopened) using the RFID circuit (21) and a remote control circuit that powers and probes the battery-less RFID circuit.

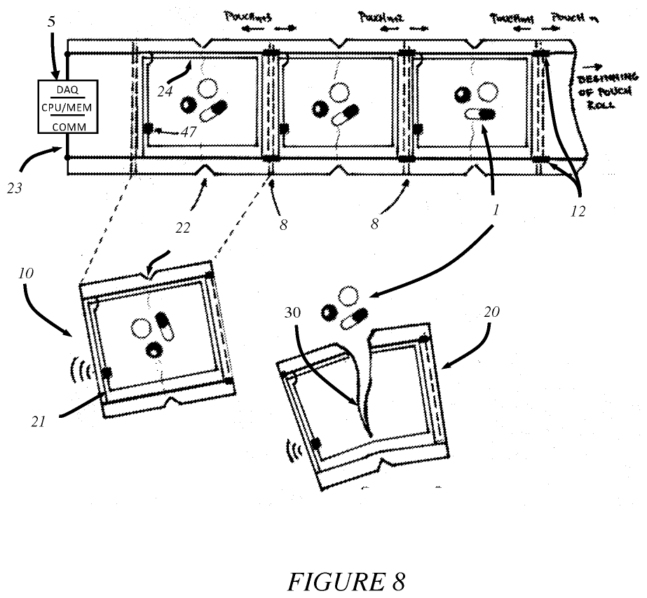

In another embodiment shown in FIG. 8, the RFID antenna/coil (24) spans a wider area on the pouch, offering improved detection range. The RFID chip (21) transmits serialization and state information when probed by a remote-control circuit.

Another embodiment that is described in FIG. 8, is one where the RFID antenna (24) acts as a receiver of ambient radiofrequency (RF) energy that is then modulated by the chip (21) and then retransmitted back from the same antenna (24) using a delay function. This creates echoes in the ambient RF energy at a specific wavelength band that can be picked up by a remote-control circuit that is tuned to this wavelength band. The echo wavelength of the unopened pouch (10) may be altered after the coil (24) has been interrupted by the tearing along the crease line (22) therefore this sudden change in the echo wavelength can be used to detect opening of the pouch (20).

In one embodiment, the remote interrogation device is a simple RF receiver and receives RF signals at a frequency used in radio communications, FM, AM, UHF or other range coming from ambient sources. The presence of an RF circuit (21) in the proximity of the interrogation device (41) will cause a reflectance of the signal, generating thus a shift in the phase of the signal that can be picked up by the interrogation device transceiver. This phase shift or reflectance may be used to detect the presence of the packaging in the proximity of a remote interrogation device (41). The printed RF circuit (21) is modulating and retransmitting the ambient RF signals, and then the remote interrogation device (41) picks up the modulated signal. The modulation depends on the inductive characteristics of the RF circuit coils/antennas (24) and thus can be decoded by the interrogation module (41) to detect state changes as well as serialization information.

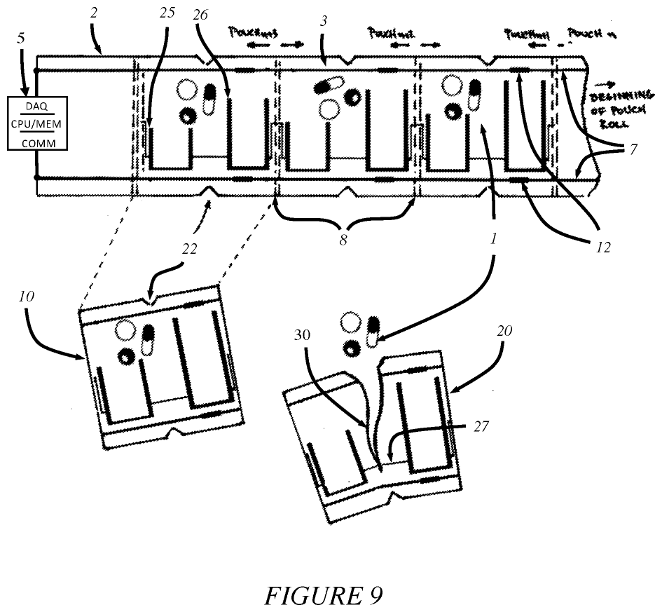

In one embodiment, shown in FIG. 9, the pouch (3) contains the medication payload (1) that may be comprised of multiple oral drugs, pills, capsules, tablets or vials, syringes, cartridges. The RFID circuit that is printed on each separated sachet comprises one or multiple inductive circuits or coils that have one or multiple resonant frequencies fi. The inductive circuits are probed using a remote interrogation device that can emit and receive RF signals and thus can determine said frequencies fi by means of resonance analysis. The RF circuit resonant frequencies fi change when said circuit is interrupted such as in the case where a sachet or a pouch or blister pack has been opened to release its contents. The new resonant frequencies fi′ are consequently probed using the interrogation device that can emit and receive RF signals. An abrupt shift in these frequencies signify the opening of the packaging and thus the release of its contents. As shown in FIG. 5, each pouch (3) holds an impendence element (12) that can be a resistor, a capacitor or an inductor, an RLC circuit. These impendence elements are connected in series or in parallel using connecting conductive or resistive lines (7) that run across the entire roll (2). Removal of one pouch from the roll (2) results in a change in the overall impendence between the two connecting lines (7) and can be measured using a control unit (5) connected at any point along the connecting lines (7). Each pouch (3) is separated from the next one by means of a crease or partial perforation (8). Each pouch holds antennas (25) and (26) that have a specific resonant frequency when inductively probed by an RF signal. This functionality is particularly useful when a pouch (10 or 20) has been removed from the roll and the above mentioned detection mechanism (impedance change along lines (12) is no longer available. Each antenna (25) and (26) has a characteristic shape pattern that causes it to resonate at different frequencies when connected and when separated. When probed by a wide band spectrum signal or a sweeping frequency signal emitted by a remote-control circuit, each coil resonates at its resonant frequency and transmits an echo back to a receiver. When the pouch is torn at the crease (22), the antenna connecting line (27) that connects the pouch antennas (25 and 26) is interrupted and the resonant frequency of the pouch antennas (25 and 26) changes. This sudden change in resonant frequency can be picked up by the remote-control circuit and interpreted as a state change for the pouch, unopened (10) and opened (20) when the payload (1) has been released.

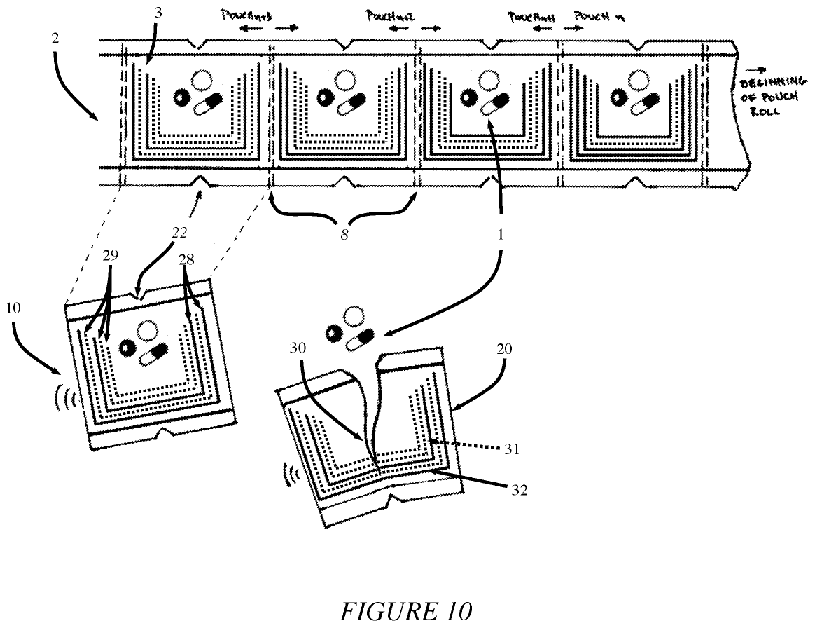

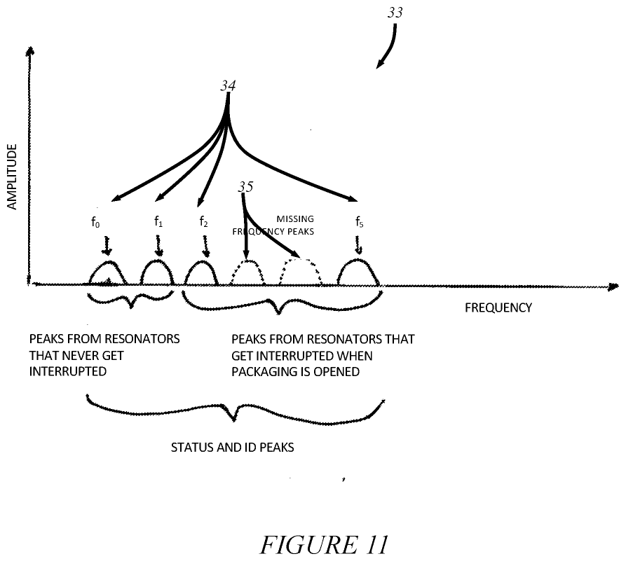

In another embodiment, shown in FIG. 10 each pouch (10 or 20) of the roll (2) holds a combination of resonators (28) that have different resonant frequencies. When a broad band signal is emitted from a remote-control circuit, each of these resonators resonates at a specific frequency. The remote interrogation device emits a broad band RF signal bound by a lower and a higher frequency range. This RF signal induces currents in the drug package's RF circuit (21) which in turn will produce an RF echo. The interrogation device (41) then receives the signal reflected back by the packaging inductive coils (24) at certain resonant frequency peaks (34). These frequencies may be interpreted as bits of digital information that can be employed to identify the serial number of the packaging. When on ore more of the drug package's inductive resonator (24) gets interrupted by a tear or a cut (30) in the package, these resonant frequencies disappear or/and appear at a different frequency range. This change can be interpreted by the remote interrogation device as a change in the state of the packaging, open or unopened. By selecting a combination of these resonators (28), a serialization pattern can be generated. In FIG. 10, the resonators are depicted in solid black (28) while dotted or gray lines depict placeholders that resonators could be printed but are omitted (29) for the given pouch (10). When the pouch (10 or 20) is torn at the crease (22), the tear (30) can propagate partially or throughout the whole width of the pouch interrupting a part of the resonators (30) leaving others, the reference echo resonators (32), intact. Crease protection can be achieved by means of tear arresting mechanism on the pouch, such as a fold or a fortification of the film locally by printing a dielectric or adding a laminated strip that will stop the crease to prevent the reference echo coils (32) from being interrupted. Once the pouch has been opened, the medication (1) can be delivered to the subject. FIG. 11 shows a frequency spectrum (33) of such a resonator system, when probed by a remote broad band or sweeping frequency signal. Solid lines represent echoes (34) generated by resonators (28), while dotted lines represent missed echoes (35) from omitted resonators (29) that are not present in the specific pouch (20). The selective pattern of resonators can be used to serialize the pouches. Each one of the frequency peaks (34 and 35) represents a single information bit. An inverse Fourier transformation of the spectrum (33) can be performed on the echo signal back from the resonators to detect the discrete peaks and each peak can serve as an information bit that can be used as a serialization information or state (opened/unopened) information. When the pouch (10) is probed, the combination of the pattern of the frequency peaks (34) and its amplitude determines the serialization information of the pouch (10) that has been torn form the roll (2). When a pouch is opened, some of these resonators are interrupted (31) by the crease resulting in the drop of the respective peaks in the power spectrum (33) while some of the resonators are protected from the crease and still remain active (32) to transmit a reference echo back to the remote-control circuit. This can determine the state of the pouch, unopened (10) or opened (20).

In one embodiment, the interrogation device (41) can emit an RF signal of narrow or broad frequency spectrum. It can also receive signal echoes, or signal reflections from the RFID circuit (21) in the proximity and decompose the signal. The interrogation device (41) has logic that allows it to discriminate dominant frequency peaks (34), shown in FIG. 11, in the signal received, by means of Fourier Transformation and Peak Detection. The interrogation device (41) typically holds one or more antennas, or coils, preferably positioned on different planes. When an inductive circuit (21) is interrogated, the interrogation device's (41) antenna or coil with the best alignment with the inductive circuit can be selected; this can be determined from the amplitude of each one of the echo signals. In one embodiment, the magnitude and direction of all the received signals is calculated by probing and receiving signal simultaneously by all the antennas or coils.

The interrogation device is located on a wearable (43) such as a smart watch or a smart ring or a smart watchband.

The control unit (5) is comprised by a data acquisition system (DAQ) (13), a processor and memory system (14) and a communication system (15) that can measure changes in resistance and/or capacitance and/or inductance and/or impedance or any combination thereof. The control unit (5) is able to communicate with a handheld device or a connected device (16) such as a smartphone, smartwatch or any other connected devices that is able to receive signals generated by the communication system (15) that transmits the events acquired from the pouch roll (2) or individual pouches (10) to the internet (17). A server (18) is processing and storing the events and presents analyzed data to a terminal front-end (19) that is connected to the internet as shown in FIG. 6.

In one embodiment, each sachet or pouch (3) holds features that upon tearing it produce a characteristic sound that can be picked up by a microphone that belongs to a wearable or a static device such as a digital assistant. Once the characteristic sound is detected, the wearable or static device may deduce that the sachet has been opened and therefore the payload medication has been released to the user. Said features may be metallic elements such aluminum foil that produce crackling sound when manipulated. Said features may be consecutive polymeric or metallic elements such as strips attached to the sachet that each produce material or thickness inhomogeneities in the path of the tear, and therefore can cause sound generation. This unique sound pattern of the packaging opening may further be refined using a machine learning algorithm that adapts the template pattern to the specific patient ambient noise and the particular patient use mode. This specific sound can be in the audible or ultrasound spectrum or both. The sound pattern can be a combination of the handling of the pouch before opening it and while opening it. Identification of pouch opening can be further improved by means of accelerometer and gyroscope readings from the wearable device that is worn in the hand or finger.

The abovementioned embodiments and examples all refer to sachet or pouch drug packaging and methods of detecting whether the package has been opened or not. However, a lot of drugs are packaged in a blister pack configuration. This disclosure hereby further describes how to apply the same subject matter of detection of the state of the package in blister pack configurations.

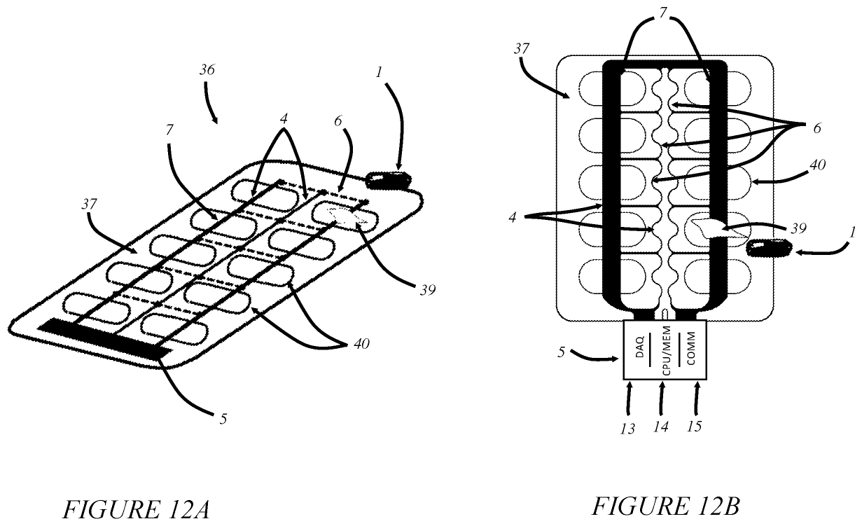

In one embodiment shown in FIG. 12, the medication packaging is a blister pack (36) comprised of a plurality of blisters (40) that are backed by a polymeric or paper or metallic or composite film (37). The blister pack (36) holds a circuit (4) on its backing film, and components of the circuit (4) get interrupted when a blister (40) is opened by means of perforation of the underlying backing film (37). This principle is similar to the disruption that occurs when a sachet is removed or torn open from a roll as described above. The change in the circuit (4) can lead to a change in its electric, resistive, impendence, magnetic or electromagnetic properties. The change can then be probed by the control unit (5) or an interrogation device (41). Such interrogation device (41) may be a wearable or static device that has an inductively or electromagnetically coupled transceiver, or an ambient RF receiver.

In one embodiment shown in FIG. 12, the medication packaging is a blister pack (36) comprised of a plurality of blisters (40) that are backed by a polymeric or paper or metallic or composite film (37). The blister pack (36) holds a circuit (4) on its backing film, and components of the circuit (4) get interrupted when the backing film (37) adjacent to a blister (40) is perforated by the medication (pill or tablet or capsule). This change in the circuit can be detected by a control unit (5) that holds an on board data/signal acquisition system (13) or DAQ. The control unit (5) may contain processing (14) and storage capabilities. It further comprises a communication module (15) that can transmit information to a remote device such as a smartphone (16). Such remote device (16) can be a Bluetooth equipped device, or a Wi-Fi equipped device, or another RF receiver equipped device. The remote device can be a smart phone, smart watch, router, digital assistant or other device.

In one embodiment shown in FIG. 12, the circuit (4) that is printed or embedded or attached on the backing film (37) of the blister pack (36) holds parallel conductive connecting lines (7) that have resistive elements (6) that span the area of backing film (37) at the area of the perforation (39) when a medication (1) is pushed out and through the backing film (37) of the blister pack (36). These resistive elements (6) get interrupted when the film (37) that holds them gets perforated due to the oral medication (1) force and hence the total resistance between the two connecting lines (7) changes. This change in resistance is detected from the control unit (5) that holds the data acquisition system (13). In one embodiment, the connecting lines (7) may turn or have curves in order to cover multiple rows of blisters.

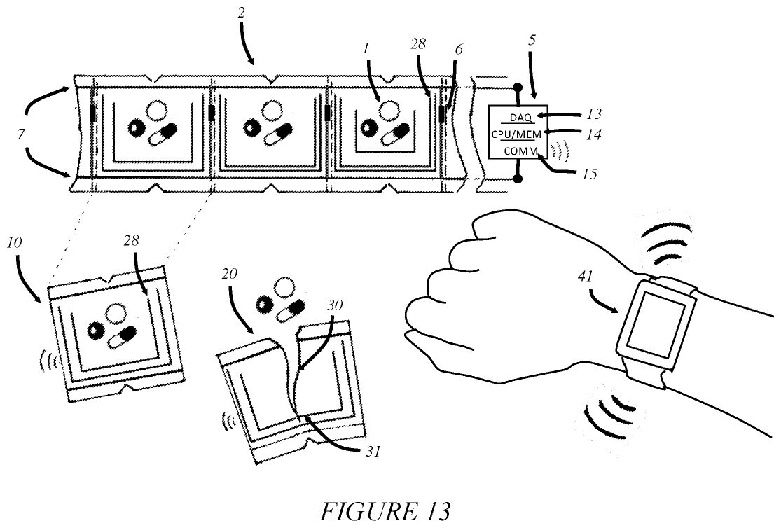

One generic embodiment shown in FIG. 13, shows a schematic of a drug package such as a roll (2) and a remote interrogation device (41) that can be included in a wearable device (43). Probing can happen in two modes: A) one mode is when the pouch is still connected to the roll (2), via an impendence or resistance monitoring between the two connecting lines (7) running along the length of the roll. The connecting lines (7) are connected at the level of each pouch with an impendence or resistive element, such as a resistor (6). The roll is equipped with a control unit (5) that includes signal/data acquisition system (13), a processing and memory system (14), and a communication module (15). Once a single pouch (10) is removed from the roll (2), the overall impendence or resistance between the two lines (7) changes. The data acquisition module (13) senses that change and the processor (14) logic determines if this change constitutes a pouch removal. The event is stored in the memory unit and the communication module (15) will transmit this information once a connected device (41) is in range. The connected device can be a smart watch, phone, computer or Wi-Fi router or other. The communication protocol can be bluetooth or Wi-Fi or other. B) The other mode of probing is for when an individual pouch (10) has been detached from the roll (2) for use at a later time and most likely at a different location, such as in the case of somebody traveling and wanting to get his medication (1) with him but not the entire roll (2). The interrogation device (41) is emitting a broadband RF signal that is received by the printed circuit or antenna (28), which in turn modulates the signal in terms of the state of the pouch (open/unopened) and in terms of serialization info. State information is achieved by means of elements of the antenna being interrupted by a tear (30) through the pouch, hence the resonant frequency and amplitude of the interrupted antenna elements change abruptly. The signal is reflected back and picked up by the interrogation device (41). The interrogation device (41) holds a receiving circuit and a processor and can monitor the signal being reflected by the open pouch (20). Changes in the received spectrum, such as resonant frequency peaks missing and/or appearing elsewhere in the spectrum are then detected. The processor of the interrogation device (41) can determine if these changes constitute a change in the state of the pouch, unopen (10) and open (20), and signal the cloud with this information as well as serialization.

In one embodiment, each sachet or pouch or blister holds printed organic LED (OLED) that can be selectively powered to turn on and alert the user of a pending dose. In one embodiment, each sachet or pouch or blister holds an electroluminescent coating that can be selectively powered to point out the pending dose to be delivered to the patient. The electroluminescent coating may be comprised by a silver or copper or organic conducting back electrode layer, a phosphorous layer, a dielectric layer and a transparent front electrode. Transparent electrodes may be comprised of indium tin oxide, or polyaniline or Poly(3,4-ethylenedioxythiophene)-poly(styrenesulfonate) or other conductive materials. These layers may be inkjet printed, or screen printed, or flexographic printed. The electroluminescence elements may be powered selectively to alert and point a user to a pending dose.

The abovementioned embodiments disclose ways to detect whether a drug package has opened and hence the medication (1) has been released from its package. In order to confirm whether the subject (42) has actually received orally the medication (1), the present invention employs means of pattern recognition on a plurality of sensors that are located on wearable (43) and static devices.

In general, a plurality of signals from wearable (43) or portable devices are processed using an Artificial Intelligence system capable of identifying patterns that can be related to the action of receiving a medication.

In one embodiment, these signals are coming from the following sources: a) microphone, b) accelerometer, c) gyroscope, d) magnetometer, e) light sensor, f) proximity sensor, g) camera, h) date/time, i) location. The signals are processed using a supervised or non-supervised pattern recognition algorithm to detect repeated patterns that signify the delivery of an oral, inhalable or injectable medication.

In one embodiment, the accelerometer and gyroscope signals from one or a combination of the following a) a smartwatch, b) a smart watchband or c) a smart ring, cumulatively called hereby wearable (43), can be used to produce a trajectory of motion of the hand (45) while a user/subject (42) is taking an oral medication (1) or pouring a cup of water to drink with the oral medication (1). This signature can be generalized after several times of repetition. In one embodiment, the signature can be used as an initial training set to create an initial set of features or a convolution kernel or to train weights of a recurring neural network to detect similar patterns in a dynamic system that continuously trains itself by using true, user-confirmed events to further build on the training set. A probability factor is generated to determine if a user has received his/her medication based on the matching of the current pattern to the previous ones that comprise the training set.

In one embodiment, the microphone signal from one or a combination of the following a) a smartwatch, b) a smart watchband, c) a smart ring, d) smart phone, e) tablet or f) digital assistant can be used to produce a sound signature of the process of drug delivery. This signature can include sounds of the drug container or package or sachet or blister or bottle opening, as well as the sounds of a glass or cup filling, and gulping or chewing sounds or even ambient sounds as the medication is consumed. Such sounds can be produced by noise producing features in the package as mentioned before. The signature can be generalized after several times of repetition. In one embodiment, the signature can be used as an initial training set to create an initial set of features or a convolution kernel or to train weights of a recurring neural network to detect similar patterns in a dynamic system that continuously trains itself by using true, user-confirmed events to further build on the training set. A probability factor is generated to determine if a user has received his/her medication based on the matching of the current pattern to the previous ones that comprise the training set.

That probability factor is further adjusted based on information received from the packaging state.

In one embodiment, the sensing of the drug delivery can further comprise a serialization method to denote the exact medication that is delivered. This can be implemented by means of serialization of the sound of the packaging opening in combination with the capturing of said sound by the wearable's microphone.

In one embodiment, the geolocation before, during and after the time of drug delivery can be tracked using a wearable GPS or Wi-Fi or Bluetooth localization. This localization pattern, before, during and after the drug delivery can be employed to generate a signature of the process of drug delivery. This signature can be generalized after several times of repetition. For example, the pattern of going to a specific area of a building, spending a specific period of time and then going to another area of the building is a pattern that repeats itself in the case of a particular subject. In the case of Wi-Fi indoor localization, the particular Wi-Fi networks available as well as their signal strength can be used to produce a unique RF phenotype of a particular room. A wearable (43) equipped with a Wi-Fi module, can thus reveal which room the user (42) is situated. This contributes to the probability that a user is receiving his/her medication based on the patterns that he/she employs. In the case of GPS localization, the gross location can also contribute to the probability that a user is receiving his/her medication based on the patterns that he/she employs. In one embodiment, the signature can be used as an initial training set to create an initial set of features or a convolution kernel or to train weights of a recurring neural network to detect similar patterns in a dynamic system that continuously trains itself by using true, user-confirmed events to further build on the training set. A probability factor is generated to determine if a user has received his/her medication based on the matching of the current pattern to the previous ones that comprise the training set. The sum of all geo-localization information is used to compile a probability function that determines if a user has received his/her medication.

In one embodiment, the light intensity that is sensed from a wearable device (43) before, during and after the time of drug delivery can be employed to create a signature of the process of drug delivery. This signature can be generalized after several times of repetition, and subsequently can be used as a convolution kernel to detect similar patterns in a dynamic system that continuously trains itself. A probability factor is generated to determine if a user has received his/her medication. The light intensity may for example indicate the conditions in a particular room where the user (42) usually receives his/her medication (1). In one embodiment, the light sensor and/or the proximity sensor can be used to produce a light intensity map of the light been scattered or reflected while a user is taking an oral medication or pouring a cup of water to drink with the oral medication. This signature can be generalized after several times of repetition. In one embodiment accelerometer data and inertia measurement are combined together with the light intensity map and are used to produce a combined signature of the said activity. The signature can be used as a training set or a convolution kernel to detect similar patterns in a dynamic system that continuously trains itself. A probability factor is generated to determine if a user has received his/her medication.

In one embodiment, the day and time of the day that the above-mentioned signatures are considered to generate a sequence signature that is tied to the process of drug delivery. In one embodiment, the signature can be used as an initial training set to create an initial set of features or a convolution kernel or to train weights of a recurring neural network to detect similar patterns in a dynamic system that continuously trains itself by using true, user-confirmed events to further build on the training set. A probability factor is generated to determine if a user has received his/her medication based on the matching of the current pattern to the previous ones that comprise the training set. A probability factor is generated to determine if a user has received his/her medication.

In one embodiment, signals from a connected drug delivery device such as a connected pill bottle, a connected insulin or drug pen, a connected auto-injector, a connected inhaler, a connected eye medication delivery device can be incorporated in the creation of a probability function that includes the sum of the abovementioned probabilities to confirm delivery of medication using statistical modeling. Each individual probability function will carry its own weight based on the user, the time, the medication type, the disease etc. These weights can be preprogrammed, or dynamically defined using Artificial Intelligence training algorithms.

All of the abovementioned signatures can be analyzed separately or cumulatively using Machine Learning pattern recognition technologies. These technologies can produce a probability factor that a medication has been received.

In one embodiment, feature computation is used to feed the pattern recognition algorithm. Some of the time and frequency domain features computed are the mean, the standard deviation, the median, the maximum, the minimum, the integral, the sum of squares, the interquartile ranges, the total entropy, the autoregression coefficients, the correlation between signal pairs, the skewness, the kurtosis, the band energies of the FFT spectrum, the angle between signals, the distance between time-frequency spectrum peaks. Said features are then used as inputs into a pattern recognition algorithm such as Support Vector Machine or k-nearest Neighbors or a Classification and Regression Tree or Naïve Bayes or Bagged Decision Tree or Random Forest or Gradient Boosting Tree that is/are using the computed features and are trained using prior labeled sets to estimate probability that the subject is receiving the medication

In one embodiment, the pattern recognition algorithm employs a plurality of the sensor signals as input in a Recurrent Neural Network that is using the raw sensor signals and that is trained using prior labeled sets to estimate probability that the subject is receiving the medication.

The abovementioned embodiments address the medication adherence problem for oral, solid medication configurations. Still there are several active pharmaceutical ingredients that are taken in liquid form, either orally, or topically, or via injection. A variation of the presented invention allows to measure the level of a liquid in a vial and is presented hereby.

In one embodiment, the packaging of the drug is comprised of a glass vial or syringe or cartridge and the drug is in liquid form. A sticker on the glass contains a printed inductive circuit that holds a sensing component. The sensing component may be a piezoelectric crystal connected in series or in parallel with the inductive circuit to form an LC circuit. The piezoelectric crystal is powered by the induction current generated in the induction circuit when it is coupled with an external control circuit that transmits an RF signal. Powering of the piezoelectric crystal will cause it to create a mechanical or acoustic signal that is propagated on the glass container by means of acoustic coupling between the crystal and the glass. The control circuit is capable of creating a sweeping frequency RF wave that then causes a sweeping frequency current in the induction circuit which in turn causes a sweeping acoustic frequency on the crystal. The amplitude of the acoustic vibration depends on the amount of liquid in the vial, and at acoustic resonance gets maximized. At resonance, the current amplitude in the LC system gets maximized and can be picked up as the dominant echo in the interrogation device. Thus, the interrogation device can remotely sense how much liquid is present in the glass container. Any changes in the resonant frequency is a quantifiable indication that a dose has been dispensed.

In one embodiment, the interrogation device transmits an RF signal that when picked up by the vial/syringe/cartridge coil generates inductive currents that in turn cause a broad frequency excitation signal that is fed to the piezoelectric crystal. The piezoelectric crystal produces a spectrum of frequencies that are reflected or absorbed according to the scattering media and the volume left in the vial. The crystal senses the reflected waves and sends back an altered RF signal that is picked up by the interrogation device. The pattern of reflected waves that is transmitted is a characteristic of the container and the volume left within the vial.

In one embodiment, said piezoelectric crystal produces a sound of broad spectrum. The sound amplitude maximizes when resonance is reached, similar to a bottle reaching Helmholtz resonance. A microphone on the interrogation device, can pick up the sound signal and identify the resonant frequency. That resonant frequency can be used to determine the amount of liquid left in the vial.

1-113. (canceled)

114. A method for detecting and confirming delivery of a medication to a subject, comprising:
i. a medication packaging that includes a feature, wherein at least one property of the feature changes in response to releasing the medication from the medication packaging;
ii. a pattern recognition algorithm operating in a controller, the pattern recognition algorithm disposed to determine a probability of the subject receiving the medication, wherein the pattern recognition algorithm determines the probability based on medication signals from the medication packaging that are indicative of the at least one property of the feature and at least one of subject signals from wearable sensors associated with the subject, ambient signals from ambient sensors disposed in a subject's environment, and contextual information;
iii. a communication device associated with the controller, the communication device disposed to transmit to an external controller information indicative of the at least one property of the feature and the probability of the subject having received the medication.

115. The method of claim 114, wherein the medication packaging is one of:
(a) a strip, a roll or a sheet that has sequential tearable or removable compartments, each compartment containing a dose of the medication, wherein the feature is an electrical property of a circuit that is part of the packaging;
(b) a blister pack that holds multiple blisters, each blister containing a dose of the medication, wherein the feature is an electrical property of a circuit that is part of the blister pack that changes in response to tearing or perforation of one of the multiple blisters;
(c) an individual sachet containing a dose of the medication, wherein the feature is an electrical or physical property of the circuit that changes in response to opening the sachet.

116. The method of claim 114, wherein the feature is an electrical property of a circuit integrated with the medication packaging, and wherein the electrical property is at least one of an electrical resistance of a node of the circuit, a capacitance of a node of the circuit, an inductance of a node of the circuit, an impedance of a node of the circuit and wherein the controller is disposed to receive the medication signals as an input signal from the circuit integrated with the medication packaging.

117. The method of claim 114, wherein the feature is a characteristic sound that is produced in response to tearing or perforating the medication packaging, and wherein the medication signals include an input signal from a microphone that is included within a wearable device disposed on the subject.

118. The method of claim 114, wherein the feature is a change in an electromagnetic property of a circuit integrated with the medication packaging in response to tearing or perforating the medication packaging, and wherein the electromagnetic property is at least one of a phase modulation index of the circuit, a frequency modulation index of the circuit, an amplitude modulation index of the circuit, a resonant frequency of the circuit, and wherein the medication signals include an input signal from a radio-frequency (RF) transceiver that is included within a wearable device disposed on the subject.

119. The method of claim 118, wherein the pattern recognition algorithm further determines the probability based on the subject signals, and wherein the subject signals include an internal property of a characteristic movement that the subject performs while dispensing or consuming the medication, which characteristic movement is indicated to the controller by an input signal from an accelerometer, a gyroscope, and/or a magnetometer that is/are included within a wearable device disposed on the subject.

120. The method of claim 119, wherein the pattern recognition algorithm further determines the probability based on a time sequence of events that are synchronously or sequentially captured through the medication signals and through the subject signals.

121. The method of claim 120, wherein the wearable device is at least one of a smart watch, a smart ring, a smart watchband, a smart wristband, a pair of smart glasses, a smart phone, and a pair of smart clothing.

122. The method of claim 114, wherein the communication device is disposed to transmit information to the external controller using a cloud computing network, wireless signals, wired signals, a local area network, or a wide area network infrastructures.

123. A medication adherence monitoring system, comprising:
i. a medication packaging that includes a feature, wherein at least one property of the feature changes in response to releasing the medication from the medication packaging;
ii. a pattern recognition algorithm operating in a controller, the pattern recognition algorithm configured to determine a probability of the subject receiving the medication, wherein the pattern recognition algorithm determines the probability based on medication signals from the medication packaging that are indicative of the at least one property of the feature, and at least one of subject signals from wearable sensors associated with the subject, ambient signals from ambient sensors disposed in a subject's environment, and contextual information;
iii. a communication device associated with the controller, the communication device configured to transmit to an external controller information indicative of the at least one property of the feature and the probability of the subject having received the medication.

124. The system of claim 123, wherein the medication packaging is one of:
(a) a strip, a roll or a sheet that has sequential tearable or removable compartments, each compartment containing a dose of the medication, wherein the feature is an electrical property of a circuit that is part of the medication packaging;
(b) a blister pack that holds multiple blisters, each blister containing a dose of the medication, wherein the feature is an electrical property of a circuit that is part of the blister pack that changes in response to tearing or perforation of one of the multiple blisters;
(c) an individual sachet containing a dose of the medication, wherein the feature is an electrical or physical property of the circuit that changes in response to opening the sachet.

125. The system of claim 123, wherein the feature is an electrical property of a circuit integrated with the medication packaging, and wherein the electrical property is at least one of an electrical resistance of a node of the circuit, a capacitance of a node of the circuit, an inductance of a node of the circuit, and an impedance of a node of the circuit, and wherein the medication signals include an input signal from the circuit indicative of a change in the electrical property of the feature.

126. The system of claim 123, wherein the feature is a characteristic sound that is produced in response to tearing or perforating the medication packaging, and wherein the medication signals include an input signal from a microphone that receives the characteristic sound and that is included within a wearable device disposed on the subject.

127. The system of claim 123, wherein the feature is a change in an electromagnetic property of a circuit integrated with the medication packaging in response to tearing or perforating the medication packaging, and wherein the electromagnetic property is at least one of a phase modulation index of the circuit, a frequency modulation index of the circuit, an amplitude modulation index of the circuit, a resonant frequency of the circuit, and wherein the medication signals include an input signal from a radio-frequency (RF) transceiver that is included within a wearable device disposed on the subject.

128. The system of claim 123, wherein the pattern recognition algorithm further determines the probability based on the subject signals, and wherein the subject signals include an internal property of a characteristic movement that the subject performs while dispensing or consuming the medication, which characteristic movement is indicated to the controller by an input signal from an accelerometer, a gyroscope, and/or a magnetometer that is/are included within a wearable device disposed on the subject

129. The system of claim 128, wherein the pattern recognition algorithm further determines the probability based on a time sequence of events that are synchronously or sequentially captured through the medication signals and through the subject signals.

130. The system of claim 128, wherein the wearable sensors are included in at least one of a smart watch, a smart ring, a smart watchband, a smart wristband, a pair of smart glasses, a smart phone, and a pair of smart clothing.

131. The system of claim 123, wherein the communication device is disposed to transmit information to the external controller using a cloud computing network, wireless signals, wired signals, a local area network, or a wide area network infrastructures.
